(19) United States
(12) Patent Application Publication
(10) Pub. No.: US 2015/0198580 A1
(43) Pub. Date: Jul. 16, 2015

(54) COVERSLIP AND METHODS FOR REMOVING

(71) Applicant: GENERAL ELECTRIC COMPANY, Schenectady, NY (US)

(72) Inventors: Brian James Grimmond, Clifton Park, NY (US); Brian Christopher Bales, Niskayuna, NY (US); Alex David Corwin, Schenectady, NY (US); Adriana Ines Larriera Moreno, Malta, NY (US); Christine Lynne Pitner, Niskayuna, NY (US)

(73) Assignee: GENERAL ELECTRIC COMPANY, Schenectady, NY (US)

(21) Appl. No.: 14/153,315

(22) Filed: Jan. 13, 2014

(51) Int. Cl. G01N 33/483

(52) U.S. Cl. CPC G01N 33/4833

(57) ABSTRACT

The invention provides a coverslip for automated decoverslipping of a tissue bearing slide comprising a horizontal base portion having a length, width, and height, at least two side wall portions extending downward from opposite sides of the base portion each having a length and width and h; and wherein the total wall volume to base volume ratio is greater than or equal to approximately 0.025.

### BACKGROUND

For multiplexed applications, tissue samples or tissue microarrays (TMA) need to be stained with many multiple molecular probes to investigate protein expression or spatial distribution quantitatively or qualitatively. Currently, the process is mostly typically performed using time-consuming iterative steps with the aid of microscopic flow cell devices followed by optical imaging and data collection.

Staining and optical imaging of the tissue samples, involve mounting the samples on slides and protection of the tissue from dehydration by application of a glass coverslip and a mounting media with refractive indices and thickness suitable for the optical hardware employed. Advanced multistage tissue analytics, such as multiplexed single tissue slide imaging, requires removal of the coverslip, or decoverslipping, in order to perform repeated operations following imaging, such as of dye inactivation or new biomarker staining. Although coverslips may be removed manually; a reliable method to enable automated decoverslipping, while maintaining optical quality and integrity of the tissue, is unavailable largely because of mounting media viscosity and the surface interactions between glass slide and coverslip which causes coverslip breakage or tissue damage.

Thus there is a need to have reliable decoverslipping as part of multistage tissue analytics process that includes multiplex staining and optical imaging, while maintain tissue integrity and optical quality.

### BRIEF DESCRIPTION

The invention generally relates to a coverslip design with a combination of width and thickness suitable for multiplex staining, optical imaging and automated decoverslipping.

In a first aspect, the invention provides a coverslip for automated decoverslipping of a tissue bearing slide comprising a horizontal base portion having a length, width, and height, at least two side wall portions extending downward from opposite sides of the base portion each having a length and width and h; and wherein the total wall volume to base volume ratio is greater than or equal to approximately 0.025.

In a second aspect, the described coverslip is a consumable component of an analytical device that is capable of staining and imaging tissue samples in multiple rounds. In certain embodiments, the analytical device comprises robotics and wherein the coverslip may be applied or removed by robotic means using a vacuum sealing device or precision clamps.

### DRAWINGS

These and other features, aspects, and advantages of the present invention will become better understood when the following detailed description is read with reference to the accompanying drawings in which like characters represent like parts throughout the drawings, wherein:

FIG. 1 is illustration of a coverslip comprising a generally horizontal base portion having and at least two side wall portions, extending downward from opposite sides of the base.

FIG. 2 is an illustration of a coverslip wherein the side wall portions are disposed along the longer edge of the base portion.

FIG. 3 is an illustration of a coverslip wherein the side wall portions are disposed along all four edges of the base portion.

FIG. 4 is a graphical representation of optical profilometry across six points of an N1 coverslipped slide shows nonuniform height and gravitational settling after 24 hours

FIG. 5 is a graphical representation of focus measure by a ratio of Brenner gradients for various coverslips, compared to N1 standard.

FIG. 6 are images showing extra mounting media to separate the microscope slide and coverslip leads to focusing issues and a loss of image sharpness for N2 coverslips.

### DETAILED DESCRIPTION

To more clearly and concisely describe and point out the subject matter of the claimed invention, the following definitions are provide for specific terms, which are used in the following description and the appended claims.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques

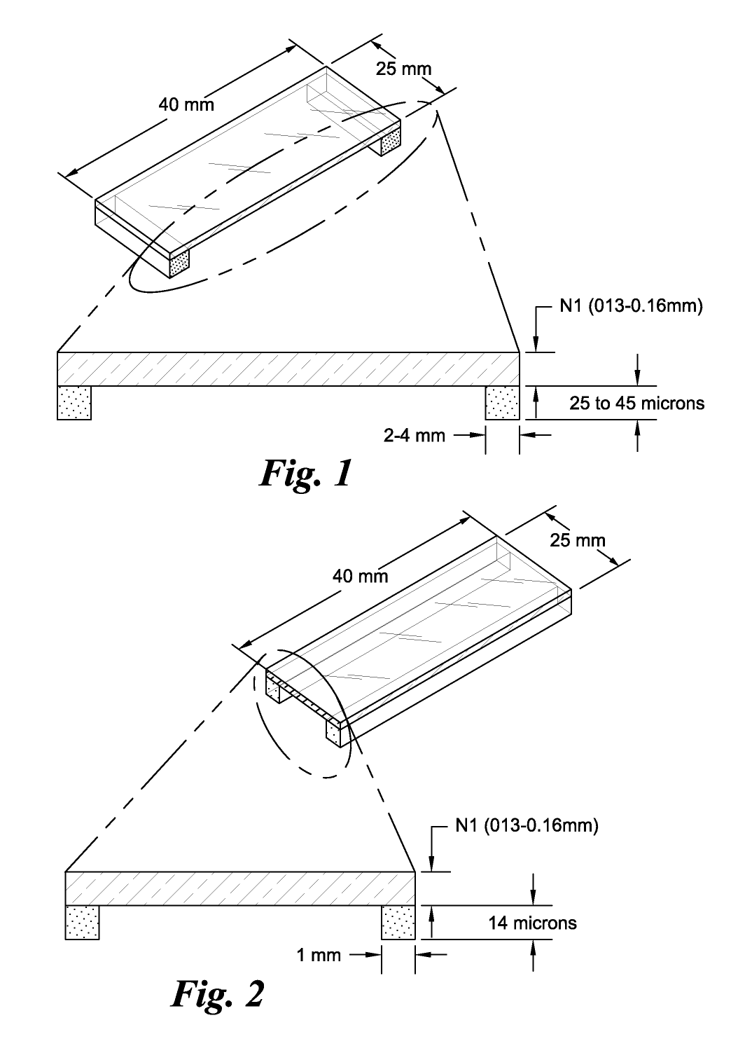

As shown in FIG. 1, in accordance with one embodiment, a coverslip is described comprising a generally horizontal base portion having dimensions of length, width, and height and at least two side wall portions, each having dimensions of length, width, and height, extending downward from opposite sides of the base portion. The introduction of the wall portions was found to reinforce the base portion sufficiently to prevent slip breakage when the coverslip is used in multistage tissue analytics, such as multiplexed tissue staining and imaging, which requires removal of the coverslip during the process. As used herein, the term “tissue” or tissue sample” refers to a sample obtained from a biological subject, including sample of tissue or fluid origin obtained in vivo or in vitro. Such samples may be, but are not limited to, a whole cell, tissues, fractions, and cells isolated from mammals including, humans, blood samples in whole or in part, as well as other biological fluids. The tissue sample may be mounted or fixed onto a solid support for example a tissue section or blood smear fixed to a microscope slide or a tissue microarray. The tissue may be that harvested for diagnosis, prognostics or therapeutic purposes such as a biopsy tissue sample.

By eliminating breakage, the coverslips may be used in an automated system for the process such as removal by a vacuum assembly robotics system. The additional stiffness also prevented slip breakage during the automated transport to the tissue wherein a coverslip is reapplied and future improves storage of the slides by providing coverslip separation such that the coverslips do not adhere to each other and can be more easily introduced into the robotics process.

The size and dimension of the cover slips may vary in width, length, and thicknesses. Coverslips are usually sized so as to fit well inside the boundaries of the microscope slide, which typically measures 25 by 75 mm. Square and round slips are usually 20 mm wide or smaller. Rectangular slips measuring up to 24 by 60 mm are commercially available and the thickness identified by numbers: No. 0-0.085 to 0.13 mm thick, No. 1-0.13 to 0.16 mm thick, No. 1.5-0.16 to 0.19 mm thick, No. 1.5 H-0.17 to 0.18 mm thick, No. 2-0.19 to 0.23 mm thick, No. 3-0.25 to 0.35 mm thick, and No. 4-0.43 to 0.64 mm thick. Thickness selection is important for high-resolution microscopy wherein biological microscope objectives are typically designed for use with coverslips in the range of No. 1 to No. 2. Coverslips that deviate from the intended range may result in spherical aberration and a reduction in resolution and image intensity. Microscope objectives may have correction collars that permit a user to accommodate for alternative coverslip thickness if necessary.

Referring further to FIG. 1, the embodiment shows an example of a typical coverslip having a dimension of 25×40 mm. The height of the coverslip may vary but as shown is between 0.13 to 0.16 microns (N1). The wall portions are disposed along the 25 mm side and have a base width of approximately 2 mm to 4 mm and a height of approximately 25 to 45 microns.

FIG. 2 is an illustrative example of an embodiment where in the side walls are disposed along the 40 mm side. In this embodiment as shown a coverslip having a N1 dimension is used however the side walls have a height of 14 microns and a base width of 1 mm.

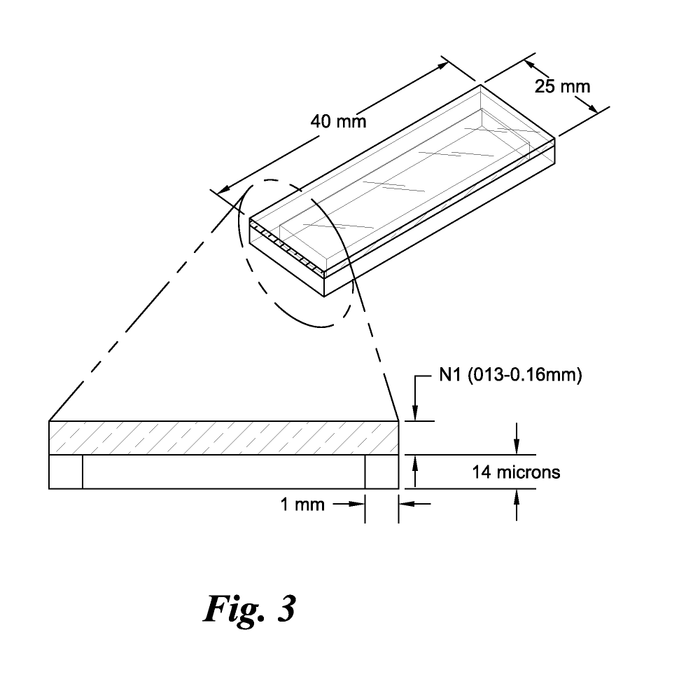

FIG. 3 is an illustrative example of an embodiment wherein the side walls are disposed along both edges of the coverslip to create a well. In the example the base width of the wall is approximately 1 mm and the thickness approximately 14 microns.

In a preferred embodiment, the wall portions are disposed along the shorter side of the coverslip. By occupying less space, more space is area is available for imaging the tissue sample and as such tissue viewing is preserved.

In certain embodiments, the dimension of the wall portion and the base portion of the coverslip is designed such that the ratio of wall volume to base volume is greater than or equal to approximately 0.025. It is understood that the ratio of being greater than or equal to approximately 0.025 may be limiting in an upper range in that large area side walls would have the disadvantage of reducing the surface area of the slide for viewing and change the focal distance. As such the ratio of wall volume to base volume is more preferably greater than or equal to approximately 0.025 to 0.05 and most preferably greater than or equal to approximately 0.03 to 0.04. At the preferred ratio the design provides a structural stiffness to the base portion which allows it to be used in an automated process without breakage due to the force acting upon the base portion when a robotic portion contacts the base. The ratio is calculated by measuring total volume of the side walls for example for embodiments having two walls on opposite ends divided by the volume of the base portion:

2(length_{wall}×width_{wall}×height_{wall})/(length_{base}×width_{base}×height_{base}).

The preferred ratio is defined further in Table 1, with coverslips having various wall and base volumes. A set number of coverslip was removed using an automated process which is defined in more detail in the experimental section. Those slides which could be removed completely initially or a second attempt, without breakage, were considered to pass. The values obtained in the table for example were calculated as such: 22×40 m:area 880, volume 132 (880 times 0.15, the average N1 slip thickness), 25×40 mm:area 1000, volume 150 (1000*0.15)

TABLE 1
Results of Side Wall Dimensions vs. Performance

| Side wall | Side wall | Side wall | Coverslip | Coverslip | Ratio | |
|---|---|---|---|---|---|---|
| Dimension | area | Volume | area | Volume | wall/CS | Pass/Fail |
| 1 mm 40 × 22 mm 14 um | 124 | 1.736 | 880 | 132 | 0.013 | F |
| 1 mm 40 mm 14 um | 80 | 1.12 | 880 | 132 | 0.008 | F |
| 2 mm 25 mm 28 um | 100 | 2.8 | 1000 | 150 | 0.019 | F |
| 4 mm 25 mm 25 um | 200 | 5 | 1000 | 150 | 0.033 | P |
| 2 mm 25 mm 35 um | 100 | 3.5 | 1000 | 150 | 0.023 | F |
| 2 mm 25 mm 45 um | 100 | 4.5 | 1000 | 150 | 0.030 | P |

In certain embodiments, the walls also prevent drift or movement of the coverslip during staining or imaging. Drift occurs due to flexing or settling of the coverslip when positioned over the tissue sample. In certain instances this drift may not be uniformed across the base of the coverslip and may vary with time. This is especially burdensome when staining and obtaining images in multistage tissue analytics as coverslip drifting results in focus drift and the inability of the microscope to maintain a selected focal plane over time. This may also be viewed as a diaphragm effect wherein the coverslip flexes with the introduction of staining solutions or an imaging media, or due to the mechanical instability from the imaging objective. The presence of the walls reduces the drift by reducing the flexibility of the coverslip.

In certain embodiments, the coverslip having two side walls is preferred. During multistage tissue analytics, such as multiplexed single tissue slide imaging, which requires removal of the coverslip, or decoverslipping, in order to perform repeated operations following imaging, such as of dye inactivation or new biomarker staining, the two side walls are sufficient to create a fluidic chamber environment equivalent in performance to a traditional well structure or wherein the coverslip acts to totally enclose the tissue sample. By being able to retain the staining solutions or other reagents in contact with the tissue sample, the desired staining or reactions can occur in a control, uniform manner. Furthermore, by having open sides, the movement of staining solutions or reagents can occur easily allowing for the tissue sample to be bathed or washed completely without the concern of trapped reagents and provide a more uniform flow across the tissue surface.

In certain embodiments, the required height of the side walls may be determined based in part on the thickness of the sample and the amount of staining solutions or reagents used. Where the sample is a tissue section, it may have a thickness between about 1 μm to about 100 μm. In some embodiments, the tissue section may occupy up to a 25 mm by 50 mm area. This results in a small internal cell volume or holding capacity of the coverslip in the range of 1 μL to 1000 μL, preferably, 25 μL to 200 μL. As such, in certain embodiments the coverslip may be designed differently for different sample dimensions to minimize the internal cell volume while still enclosing the sample and allowing the introduction of the necessary staining solutions and reagents. In certain embodiments, the dimensional tolerance may be related to the automated device or the control of reagent volume. For example in certain embodiments, the dimensional tolerance of the wall width or height may be ±10 μm. In other embodiments, the tolerance may ±6.25 μm, in still other embodiments; the tolerance may be ±5 μm. The tolerance is such that it may further aid the use of the automated device.

In certain embodiments, the coverslip base is optically transparent in a specified range of wavelengths. In an embodiment wherein the used for multiplexed tissue staining and analysis, using both a transparent coverslip and solid support allows for both epi-fluorescence imaging and transmitted brightfield imaging. This enables analysis of fluorescence-based molecular pathology as well as conventional brightfield imaging based on, for example, diaminobenzidine (DAB) staining or hematoxylin and eosin stain (H&E) chromogenic staining.

In certain embodiments, the coverslip comprises fused quartz, glass such as silicate or borosilicate glass, or. Specialty plastics of the correct optical transparency may also be used.

In certain embodiments, the coverslip may be comprised of a standard glass base which is patterned with a hydrophobic epoxy material using a screen printing process to form the walls. In still other embodiments the side walls may be a polymeric strip, glass strip or tape that is sized and capable of adhering to the base material. The side wall portion and the base material may be adhered to each other using a variety of processes. As used herein the term “adhered” or “capable of adhering” refers to joining components or materials together to form a seal at the interface of the materials. Adhering may refer to the use of a chemical adhesive to form a bond, wherein the chemical adhesive includes but is not limited to silicones, epoxies, acrylics, room temperature vulcanizing materials (RTVs), thermoplastics, or a combination thereof. Adhering may also be accomplished by overmolding one material over another to create a seal due to mechanical or chemical interactions at the interface of the two materials. In certain embodiments adhering may be accomplished through the application of external conditions such as pressure, temperature, or exposure to light or radiation. Adhering may result in a strong bond at the interface such that cohesive failure occurs at separation. In other cases, adhering may result in a bond at the interface which may be broken with a minimum amount of force such that the interface may be repositioned or the bond may be considered a temporary bond.

In certain embodiments, the coverslip may be deemed a consumable material. As used herein, the term “consumable” refers to a disposable component that is designed for a single or limited use. In some situations the consumable may have a useful life that is less than that of the system with which it is used in, in other situations, the consumable may be a part, stored and manufactured separate from the system for which it is intended to be used.

In one embodiment, a method is described wherein the coverslip described in used in an automated process involving multistage tissue analytics, such as multiplexed tissue staining and imaging as described in US patent application US2009253163A1, and U.S. Pat. No. 7,629,125. As such, in one of the embodiments, the coverslip may be may be incorporated as consumable components of an analytical device such as an automated high-throughput system that is capable of staining and imaging TMAs in one system and still further analyzes the images. As such, in one embodiment, the system is capable of illuminating the sample and capturing digital images using various optical systems including those outside the range of autofluorescence such as brightfield imaging. The system comprises robotics that are capable of positioning the tissue sample which is adhered to a solid support for multiple rounds of imaging and staining. Between the rounds of imaging and staining the coverslip may be applied or removed by robotic mean using a vacuum sealing device or precision clamps.

### EXPERIMENTAL

### Performance Comparison Raised Coverslips

Raised coverslips facilitate the automated decoverslipping of a tissue bearing slides and enable multiplexed optical imaging of tissue. Microscope slides were loaded onto an Xmatrx™ autostainer (BioGenex Laboratories, Fremont Calif.) and equal volumes (30-50 uL) of mounting media suitable for multiplexed optical imaging were applied to the slides. Standard or customized raised slips of varying hydrophobic designs and thicknesses were mounted on the slides using the vacuum pump and suction cup assembly within the Xmatrx robotic head. The slides were removed from the autostainer and hand-cleaned with damp tissues before being inverted for 30 minutes to simulate optical imaging. The slides were returned to the Xmatrx slide racks and stored for 12 hours. Automated decoverslipping of up to 40 slides was performed to a maximum of four times each, recording the number of attempts necessary to remove the coverslip, if successful. Table 2 as shown below provides detailed results from the data provided in Table 1. N refers to the number of coverslips tested; decoverslip refers to the number of coverslips removed without breakage initially or in a second attempt.

TABLE 2

| Wall | Wall edge | Wall | | Mount | |
|---|---|---|---|---|---|
| depth | length | thickness | N | volume (uL) | Decoverslip¹ |
| 0 | 0 | 0 | 5 | 30 | 0 |
| 1 mm | 40 mm | 14 um | 10 | 50 | 7 |
| 1 mm | 40 mm | 14 um | 10 | 50 | 8 |
| 1 mm | Full Border | 14 um | 10 | 50 | 8 |
| 2 mm | 25 mm | 28 um | 10 | 50 | 9 |
| 4 mm | 25 mm | 25 um | 5 | 50 | 5 |
| 2 mm | 25 mm | 28 um | 5 | 50 | 2 |
| 2 mm | 25 mm | 35 um | 10 | 30 | 10 |
| 2 mm | 25 mm | 45 um | 10 | 30 | 10 |
| 4 mm | 25 mm | 25 um | 5 | 50 | 5 |
| 4 mm | 25 mm | 25 um | 10 | 30 | 10 |
| 4 mm | 25 mm | 25 um | 5 | 30 | 5 |
| 4 mm | 25 mm | 25 um | 10 | 30 | 10 |
| 4 mm | 25 mm | 25 um | 15 | 30 | 15 |
| 4 mm | 25 mm | 25 um | 28 | 30 | 28 |
| 4 mm | 25 mm | 25 um | 39 | 30 | 39 |

¹N1 coverslips

As shown in Table 2, standard coverslips could not be removed from the slides in an automated manner and walls having a 1 mm wide pattern of less than 20 um thickness were also unsuccessful. Increasing the pattern width to 2 mm and 25 um thickness was more successfully but required multiple attempts to reliably complete the decoverslipping operation. Surprisingly, a wider 4 mm patterned strip of the same 25 um thickness proved to be a 100% reliable design for robust decoverslipping. Further increasing the thickness of the 2 mm designs to 35 um and 45 um enabled increasingly reliable decoverslipping of the narrower patterning and indicated that select assemblies of varying pattern height and thickness could be developed to reliably remove coverslips.

### Effect of Direct Contact Coverslipping on Spacing

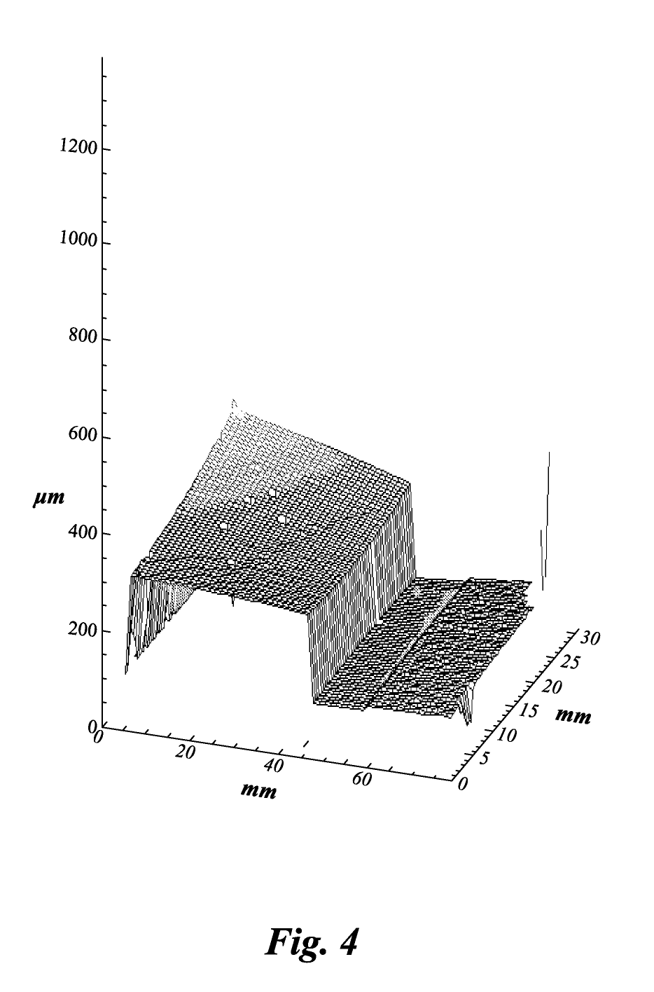

The rate of successful automated decoverslipping decreases when a standard N1 or N2 slide is handled or stored in a static position over a period of hours. Optical profilometry measurements at six points spanning the length and breadth of the slide indicated that the overall separation of slip and slide decreases over 24 hours. The decrease in the spacing between the glass surfaces as the slip gravitationally settles on the slide likely contributes to the reduced decoverslip rate. This is shown in FIG. 4 where the optical profilometry across six points of an N1 coverslipped slide shows nonuniform height and gravitational settling after 24 hours. This is also shown in Table 3 which provides the change in height of the six points after a 24 hour period.

TABLE 3
Lowering of Z-Heights (microns) from
1 h to 24 h for an N1 Coverslip

| Point | t = 0 | t = 24 |
|---|---|---|
| a | 160 | 153 |
| b | 160 | 152 |
| c | 174 | 150 |
| d | 254 | 160 |
| e | 243 | 158 |
| g | 244 | 159 |

### Effect of Coverslip Dimensions on Focusing.

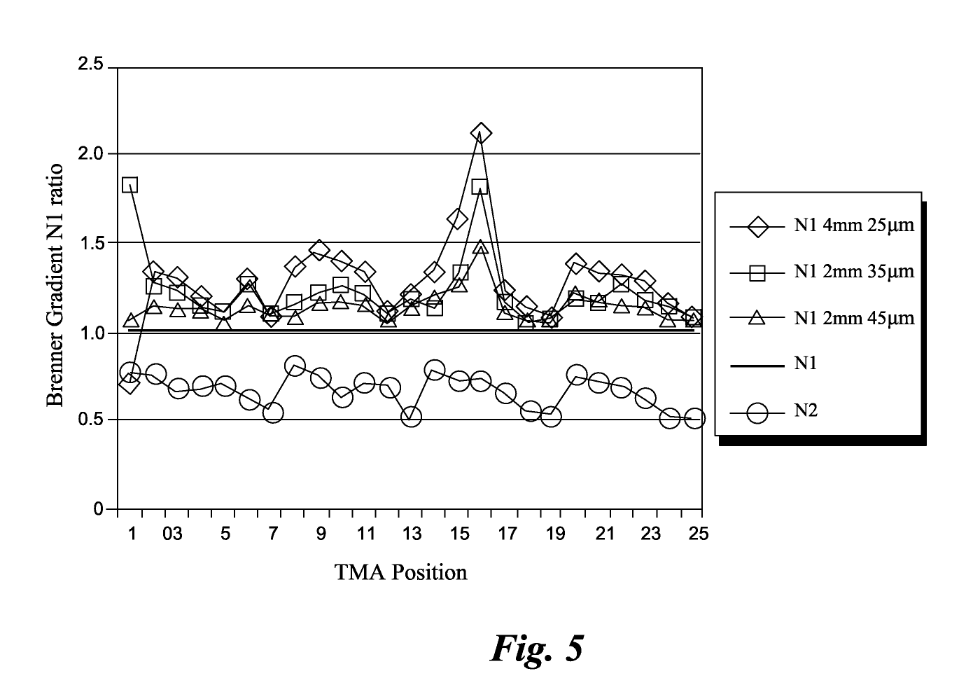

Raised Coverslips suitable for multiplexed optical imaging of tissue do not adversely affect microscope focusing and thereby image sharpness. Autofocusing algorithms determine the relationship between the microscope stage and the slide by using a focus measure to determine the local image sharpness. One focus measure is the Brenner gradient, a fast edge detector, measuring the change in intensity between neighboring pixels. As shown in FIG. 5, a higher focus measure indicates increasing image sharpness. For the Dapi channel of a stained UNC 241 TMA (Pantomics, Richmond, Calif.), the ratio of the Brenner gradient of the raised N1 coverslips to the N1 standard was generally greater than or equal to one across the majority of tissue positions throughout the multivariate universal tissue control. This indicated that the sharpness of images collected using any of the raised N1 coverslips was maintained for multiple tissue types. However, for the thicker N2 coverslip, the Brenner gradient was less than the N1 control, indicating a loss in overall image sharpness. This suggested that focusing limitations for the current optical hardware occurred with a coverslip thickness in the range of 190-230 um. This is shown further in Table 4 by the actual image quality obtained the same 20× cellular image.

TABLE 4
Image Quality obtained using the 20X cellular image

| | N1 4 mm | N1 2 mm | N1 2 mm | | |
|---|---|---|---|---|---|
| | 25 um | 35 um | 45 um | N1 | N2 |
| Av | 1.28 | 1.23 | 1.14 | 1 | 0.67 |
| s.d. | 0.25 | 0.19 | 0.09 | 0 | 0.09 |

### Effect of N2 Coverslip on Image Quality

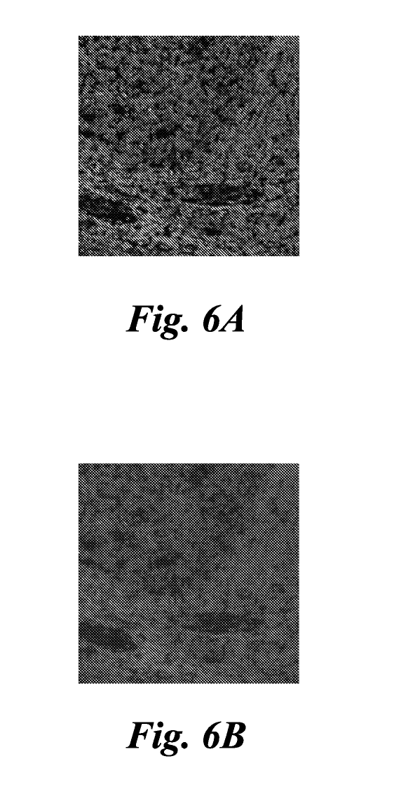

In a further study, using larger volumes of mounting media (80 uL) and standard slips could be used to enable the automated removal of coverslips. However, the failure rate (50% N1 slips, 20% N2 slips) was too large to allow automated multiplexed imaging. Additionally, the use of extra mounting media to separate the microscope slide and slip leads to focusing issues and a loss of image sharpness for N2 slips in particular. The images obtained are shown in FIG. 6.

### Comparision to Lifter Slips

In a further study, commercially available raised coverslips, called Lifter Slips, were evaluated for the automated removal of coverslips. However, the 60% failure rate observed for these devices during the decoverslipping process was too large to be suitable for reliable automated multiplexed imaging. Lifter Slips are coverslips designed with side wall portions along the two longer edges of the base to facilitate hybridization of DNA reagents on slides. The dimensions of the Lifter Slips (Thermo Scientific 22×40, N2 with 1 mm wall width and 30 um thickness) were measured using a digital micrometer and calipers to reveal a wall:coverslip volume ratio of 0.0249. This value is below the described ratio for reliable decoverslipping and is a likely factor in the poor decoverslipping performance of these devices.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A coverslip for automated decoverslipping of a tissue bearing slide comprising:
a horizontal base portion having a length, width, and height;
at least two side wall portions extending downward from opposite sides of the base portion each having a length and width and h; and
wherein the total wall volume to base volume ratio is greater than or equal to approximately 0.025.

2. The coverslip of claim 1 wherein the wall volume to base volume ratio is greater than or equal to approximately 0.025 to 0.05.

3. The coverslip of claim 1 wherein the wall volume to the base volume ration is greater than or equal to approximately 0.03 to 0.04.

4. The coverslip of claim 1 wherein the two side wall portions are disposed on the narrower sides of the base portion.

5. The coverslip of claim 1 wherein the side wall portions are on all four sides of the base portion.

6. The coverslip of claim 1 wherein the side wall portions and the base portion adhere by screen printing of the side wall portions onto the base portion.

7. The coverslip of claim 1 wherein the base portion has a thickness of approximately 0.13 to 0.23 mm.

8. The coverslip of claim 7 wherein the base portion has a thickness of approximately 0.13 to 0.19 mm.

9. The coverslip of claim 1 wherein the base portion has a thickness of approximately 0.13 to 0.19 mm and each of the side walls have a width of approximately 2-4 mm, a height of approximately 25 to 45 microns and are disposed on the narrower edge of the base portion.

10. The coverslip of claim 9 wherein the base portion and side wall portions provides a holding capacity of the coverslip in the range of 1 μL to 1000 μL.

11. The coverslip of claim 10 wherein the holding capacity is in the range of 25 μL to 200 μL.

12. The coverslip of claim 1 wherein the coverslip is a consumable component of an analytical device that is capable of staining and imaging tissue samples in multiple rounds.

13. The coverslip of claim 12 wherein the analytical device comprises robotics and wherein the coverslip may be applied or removed by robotic means using a vacuum sealing device or precision clamps.
